(19) United States
(12) Patent Application Publication
(10) Pub. No.: US 2024/0091188 A1
(43) Pub. Date: Mar. 21, 2024

(54) Dosing Protocols for Administering Deuterated Arachidonic Acid or a Prodrug Thereof

(71) Applicants: Peter MILNER, Los Altos, CA (US); Mikhail Sergeevich SHCHEPINOV, Los Altos, CA (US); Retrotope, Inc., Los Altos, CA (US)

(72) Inventors: Peter Milner, Los Altos, CA (US); Mikhail Sergeevich Shchepinov, Oxford (GB)

(73) Assignee: Retrotope, Inc., Los Altos, CA (US)

(21) Appl. No.: 18/275,965

(22) PCT Filed: Feb. 4, 2022
(86) PCT No.: PCT/US2022/015366

§ 371 Date: Aug. 4, 2023

Related U.S. Application Data
(63) Continuation of application No. 17/391,909, filed on Aug. 2, 2021, now Pat. No. 11,491,130.
Application No. 17/391,909 is a continuation-in-part of application No. 17/169,271, filed on Feb. 5, 2021, now Pat. No. 11,351,143.
Application No. 17/169,271 is a continuation-in-part of application No. 17/169,271, filed on Feb. 5, 2021, now Pat. No. 11,351,143.
(60) Provisional application No. 63/177,794, filed on Apr. 21, 2021.

(51) Int. Cl. A61K 31/202; A61K 31/232; A61P 25/28

(52) U.S. Cl. CPC A61K 31/202; A61K 31/232; A61P 25/28

(57) ABSTRACT

Disclosed are methods or dosing protocols for administering deuterated arachidonic acid or a prodrug thereof to a patient in order to accelerate the time required to achieve of a therapeutic concentration of 13,13-D2-arachidonic acid in vivo.

### CROSS-REFERENCE TO RELATED APPLICATIONS

This application claims priority to U.S. patent application Ser. No. 17/169,271, filed on Feb. 5, 2021; U.S. patent application Ser. No. 17/391,909, filed Aug. 2, 2021; and U.S. Provisional Patent Application No. 63/177,794, filed Apr. 21, 2021, each of which is incorporated herein by reference in its entirety.

### TECHNICAL FIELD

Disclosed are methods or dosing protocols for administering deuterated arachidonic acid or a prodrug thereof to a patient in order to accelerate the time required to achieve a therapeutic concentration of 13,13-D2-arachidonic acid in vivo. By accelerating or shortening the period of time required to achieve a therapeutic concentration in vivo, a patient suffering from a neurodegenerative disease retains more of his or her functionality as compared to untreated patients.

In one embodiment, the dosing protocols or methods are applicable to neurodegenerative diseases such as Amyotrophic Lateral Sclerosis (ALS). The methods include administering to a patient suffering from ALS a composition comprising a deuterated arachidonic acid, a deuterated arachidonic acid ester, or a prodrug of either using a dosing regimen that provides for in vivo concentrations of deuterated arachidonic acid at a level where the progression of the disease is markedly reduced.

### BACKGROUND

Neurological diseases and their corresponding pathological hallmarks reflect the degeneration and death of neurons with the corresponding loss of functionality (i.e., either cognitive or muscular). Once diagnosed, most patients undergo a rapid rate of disease progression often resulting in death.

There are no cures for these diseases. Rather, current therapies attempt to slow the rate of loss of functionality in treated patients. Indeed, there are now standardized tests for most of these diseases that evaluate the patient's functionality as the disease progresses and then correlates the extent of loss between two time points. In the case of ALS, the rate of disease progression is measured by evaluating a set of criteria set forth in the “ALSFRS-R” tests. In the absence of therapy, the rate of loss of functionality is referred to as the “natural history” of the disease. At a later date, a further evaluation is conducted, and the loss of functionality is determined between the two evaluations which is then converted into a percent loss. Generally, the therapies to date have shown little impact on the rate of loss of functionality in the treated patients. For example, a recent report showed that a two-drug combination administered over a 24-week period to the treated group provided for an overall improvement in the ALSFRS-R score of 2.4 units compared to the rate of disease progression in the untreated group. This report further stated that it was equivalent to 6 weeks of less progression of the disease and was considered to be an exciting clinical result. See, e.g., sitn.hms.harvard.edu/flash/2020/slowing-als-with-a-two-drug-therapy/.

A common factor in the disease pathology of neurodegenerative diseases is the oxidative damage of the lipid components of neurons caused by reactive oxygen species (ROS). This damage is the result of oxidative stress created by an imbalance between routine production and detoxification of ROS which leads to oxidative attack on the lipid membrane of cells. The lipid membrane as well as the endoplasmic reticulum and mitochondria of neurons is highly enriched in arachidonic acid (a 20-carbon chain polyunsaturated fatty acid (“PUFA”) having 4 sites of cis-unsaturation). Separating each of these 4 sites are 3 bis-allylic methylene groups. These groups are particularly susceptible to oxidative damage due to ROS, and to enzymes such as cyclooxygenases, cytochromes and lipoxygenases, as compared to allylic methylene and methylene groups.

Moreover, once a bis-allylic methylene group in one arachidonic acid is oxidized by a ROS, a cascade of further oxidation of other arachidonic acid groups in the lipid membrane occurs. This is because a single ROS generates oxidation of a first arachidonic acid component through a free radical mechanism which, in turn, can oxidize a neighboring arachidonic acid through the same free radical mechanism which yet again can oxidize another neighboring arachidonic acid in a process referred to as lipid chain auto-oxidation. The resulting damage includes a significant number of oxidized arachidonic acid components in the cell membrane.

Oxidized arachidonic acids negatively affect the fluidity and permeability of cell membranes in motor neurons. In addition, they can lead to oxidation of membrane proteins as well as being converted into a large number of highly reactive carbonyl compounds. The latter include reactive species such as acrolein, malonic dialdehyde, glyoxal, methylglyoxal, etc. (Negre-Salvayre A, et al. Brit. J. Pharmacol. 2008; 153:6-20). But the most prominent products of arachidonic acid oxidation are alpha, beta-unsaturated aldehydes such as 4-hydroxynon-2-enal (4-HNE; formed from n-6 PUFAs like LA or AA), and corresponding ketoaldehydes (Esterfbauer H, et al. Free Rad. Biol. Med. 1991; 11:81-128).

These reactive carbonyls cross-link (bio)molecules through Michael addition or Schiff base formation pathways and have been implicated in a large number of pathological processes such as age-related and oxidative stress-related conditions, and aging.

While it is known that oxidative stress of PUFAs contributes to some extent to the pathogenesis of a number of different diseases including neurological diseases, the underlying etiology of each such disease is so different that it is not possible to predict that a treatment providing positive results for one such oxidative mediated disease is also applicable to another such disease. Still further, given that the vast majority of ALS patients suffer from sALS where the underlying etiology is unknown, the applicability of a given treatment protocol across a spectrum of potentially divergent etiologies for ALS is unpredictable.

### SUMMARY

Heretofore, the treatment of a variety of neurodegenerative diseases including ALS employed compositions containing deuterated 11,11-D2-linoleic acid or an ester thereof, including those in a lipid bilayer form. See, e.g., WO 2011/053870, WO 2012/148946, and WO 2020/102596. Each of these references disclosed the in vivo conversion of a portion of 11,11-D2-linoleic acid or ester thereof administered to a patient to 13,13-D2-arachidonic acid which was then incorporated into the motor neurons to stabilize these neurons from oxidative damage. As such, 11,11-D2-linoleic acid or an ester thereof is one example of a prodrug of 13,13-D2-arachidonic acid.

However, when the active agent, deuterated 13,13-D2-arachidonic acid is delivered as a prodrug, e.g. 11,11-D2-linoleic acid or an ester thereof, a substantial period of time is required to reach therapeutic levels in vivo. For instance, only a portion of the 11,11-D2-linoleic acid or ester thereof is bioconverted to 13,13-D2-arachidonic acid with the specific amount being dictated by the patient's physiology as well as by the level of fat intake in the diet. The greater the fat intake, the lesser amount of this prodrug is converted into 13,13-D2-arachidonic acid. Still further, even under ideal circumstances, only about 10 to about 15 percent of the 11,11-D2-linoleic acid or ester thereof is bioconverted.

Under these circumstances, the in vivo generation of therapeutic levels of 13,13-D2-arachidonic acid can take months during which the patient is losing functionality as the disease progression continues. Accordingly, methods that significantly reduce the time required to achieve a therapeutic concentration of 13,13-D2-arachidonic acid in vivo are urgently needed. Such methods would allow for patients to achieve a much longer period of retained muscle functionality and likely a longer lifespan.

Most conventional drugs achieve a therapeutic concentration in vivo within hours of administration. However, the accumulation of the therapeutic 13,13-D2-arachidonic acid that is enzymatically converted in vivo from 11,11-D2-linoleic acid or esters thereof (i.e., drug) is not conventional and requires an extended period of time to achieve a therapeutic concentration in vivo. There are several factors involved in this delay. First, only about 90% of the fat, including polyunsaturated fatty acids, that is consumed by the patient is absorbed. Because the amount of drug administered is fixed, the more fat that is included in the patient's diet, the smaller the amount of drug that can be absorbed. Second, only a portion of 11,11-D2-linoleic acid is enzymatically converted to 13,13-D2-arachidonic acid which is generally set at around 10%. However, the exact amount varies from patient to patient based on their unique physiology. Finally, the 13,13-D2-arachidonic acid that is generated is systemically distributed throughout the body albeit preferentially to neurons.

Each of these variable functions by itself challenges the ability to create a dosing regimen for 11,11-D2-linoleic acid or ester thereof especially for dosing regimens that are designed to reduce the time required to reach a therapeutic concentration in vivo. When these variables are taken together, the challenge increases dramatically. However, as is apparent, the longer it takes to reach a therapeutic concentration of 13,13-D2-arachidonic acid in vivo, the greater the loss of functionality in the patient coupled with, in many cases, a shorter expected remaining lifespan.

The methods described herein address this problem. In particular, 11,11-D2-linoleic acid or esters thereof are dosed to patients pursuant to a dosing regimen that reduces the time required to achieve a therapeutic concentration in vivo. Such methods or dosing protocols comprise the administration of 11,11-D2-linoleic acid or an ester thereof using a primer or loading dose followed by a maintenance dose. The loading dose is designed to shorten the timeframe required to achieve a therapeutic concentration in vivo. The maintenance dose is a dose less than the loading dose but sufficient to maintain the therapeutic concentration in vivo once achieved.

In one embodiment, there is provided a method for accelerating uptake of 13,13-D2-arachidonic acid in vivo by dosing a patient with 11,11-D2-linoleic acid or an ester thereof in a patient wherein a portion of said 11,11-D2-linoleic acid is converted in vivo to 13,13-D2-arachidonic acid, the method comprising:
administering 11,11-D2-linoleic acid or an ester thereof to the patient with a dosing regimen that comprises a primer or loading dose followed by a maintenance dose, wherein:
a) said primer or loading dose comprises periodic administration of at least about 7 grams of 11,11-D2-linoleic acid or an ester thereof per day, wherein said primer or loading dose is continued for at least about 30 days to achieve a therapeutic concentration of 13,13-D2-arachidonic acid in vivo; and
b) subsequent to the completion of the primer or loading dose, administering said maintenance dose of from about 3 to about 6.5 gm per day of 11,11-D2-linoleic acid or an ester thereof per day thereof to maintain said therapeutic concentration of 13,13-D2-arachidonic acid in vivo.

In one embodiment, there is provided a method for accelerating uptake of 13,13-D2-arachidonic acid in vivo by dosing a patient with 11,11-D2-linoleic acid or an ester thereof in a patient wherein a portion of said 11,11-D2-linoleic acid is converted in vivo to 13,13-D2-arachidonic acid, the method comprising:
administering 11,11-D2-linoleic acid or an ester thereof to the patient with a dosing regimen that comprises a primer or loading dose and a maintenance dose thereby reducing said disease progression in said patient, wherein:
a) said primer or loading dose comprises periodic administration at least about 7 grams of 11,11-D2-linoleic acid or an ester thereof per day, wherein said primer or loading dose is continued for at least about 30 days to rapidly achieve a therapeutic concentration of 13,13-D2-arachidonic acid in vivo; and
b) subsequent to the completion of the primer or loading dose, periodically administering said maintenance dose to said patient wherein said maintenance dose is at least about 3 grams per day of 11,11-D2-linoleic acid or an ester thereof per day thereof to maintain said therapeutic concentration of 13,13-D2-arachidonic acid in vivo provided that said maintenance dose is less than said primer or loading dose.

In one embodiment, the dosing protocols described herein are particularly suitable for treating neurodegenerative diseases such as ALS. In such diseases, the progression of neurodegenerative diseases is significantly attenuated by dosing regimens that administers 11,11-D2-linoleic acid or an ester thereof.

Without being limited by any theory, the inclusion of deuterium at the bis-allylic positions of arachidonic acid stabilizes arachidonic acid against oxidative damage which, in turn, minimizes lipid peroxidation damage to the neurons caused by ROS. Still further, it has been found that when concentrations of deuterated arachidonic acid reach a therapeutic level in the neurons, the progression of such neurodegenerative diseases is significantly attenuated.

In one embodiment, the reduction in the rate of disease progression is ascertained by methods provided below wherein the delta between the rate of disease progression in the natural history is compared to that found during the therapy described herein. In both cases, the values are first annualized to a common time period and the delta is recorded as an absolute number.

In one embodiment, the percent change between the rate of disease progression occurring during the natural history of the patient and the decrease in the rate of disease progression during therapy is at least 30%, preferably at least 40%, more preferably at least 65% and most preferably greater than 70% or 80%. Accordingly, in some embodiments, methods disclosed herein provide for determining a percent reduction in the rate of disease progression by (i) determining a natural rate of disease progression in a patient or an average natural rate of disease progression in a cohort of patients, (ii) determining the rate of disease progression in the patient or cohort of patients during a period of compliance with administration of deuterated arachidonic acid, an ester thereof, or a prodrug thereof, and (iii) measuring the difference between the natural rate and the rate during the period of compliance and dividing the difference by the rate of disease progression during the natural history of the patient. The numerical value (as an absolute number) is then normalized by multiplying by 100.

Preferably, the concentration of a specific deuterated arachidonic acid found in the neurons is sufficient to provide at least a 30% reduction in the rate of disease progression in a patient. As demonstrated in the Examples below, the concentration of deuterated arachidonic acid in red blood cells can be correlated to that found in the spinal fluid from which neurons obtain their cellular components. In turn, the neurons acquire arachidonic acid from the spinal fluid and, as such, there is a direct corollary between the concentration in the spinal fluid and that in the motor neurons.

Therefore, the concentration of 13,13-D2-arachidonic in red blood cells acts as a proxy for the concentration in the motor neurons.

As shown in Example 1, the concentration of 13,13-D2-arachidonic acid found in the spinal fluid at 1 month after initiating a dosing regimen of 9 grams per day is about 8% of the total arachidonic acid found therein including the deuterated arachidonic acid. Since neurons obtain their fatty acids from the spinal fluid, the amount of 13,13-D2-arachidonic acid in the spinal fluid corresponds to that in the motor neurons. Moreover, a 3% concentration of 13,13-D2-arachidonic acid in the red blood cells has been shown to correlate to a significant reduction in the rate of disease progression. Accordingly, for the purposes of this application, it is understood that any reference to the concentration of a 13,13-D2-arachidonic acid in the red blood cells that is at or above about 3% correlates to a therapeutic dosing.

In one embodiment, whether the concentration of 13,13-D2-arachidonic acid in neurons is therapeutic can be assessed indirectly by administering a defined amount of a 11,11-D2-linoleic acid or ester thereof to a subject over a period of at least 1 month; periodically measuring the amount of deuterated arachidonic acid in red blood cells of the subject; assessing when a patient or a cohort of patients evidence therapy, correlating the concentration of 13,13-D2-arachidonic acid in red blood cells at the time therapy is reached; and correlating that concentration as a standard therapeutic concentration.

In one embodiment, provided are methods for reducing the rate of disease progression of ALS in a patient which method comprises: administering daily an effective amount of a deuterated arachidonic acid, a deuterated arachidonic acid ester, or a prodrug thereof, to reduce the rate of disease progression; wherein the concentration of deuterated arachidonic acid in the motor neurons is sufficient to reduce the rate of disease progression by at least about 30% as compared to the rate of disease progression during the natural history of the patient.

In one embodiment, the percentage of reduction in the rate of disease progression is ascertained by measuring the natural rate of disease progression in a patient or an average rate in a cohort of patients and measuring the rate of disease progression in said patient or cohort of patients during compliance with this method, measuring the delta (i.e., difference) between the two in absolute numbers, dividing the delta by the natural history, and then multiplying by 100.

In one embodiment, the deuterated linoleic acid or ester thereof is 11,11-D2-linoleic acid ethyl ester thereof.

In one embodiment, a therapeutic concentration of 13,13-D2-arachidonic acid in red blood cells is at least about 3% based on the total amount of arachidonic acid including deuterated arachidonic acid contained in the red blood cells.

In one embodiment, sufficient amounts of 11,11-D2-linoleic acid or ester thereof are administered to the patient such that the concentration 13,13-D2-arachidonic acid in the red blood cells is at least about 3% and preferably at least about 5% based on the total amount of arachidonic acid including deuterated arachidonic acid in the red blood cells.

In one embodiment, there is provided a tiered dosing regimen comprising two components. The first component takes into account the factors set forth above and delivers a primer or an accelerated dosing of deuterated arachidonic acid (including esters thereof) or a prodrug thereof. This primer dose provides sufficient amounts of 11,11-D2-linoleic acid or an ester thereof are to be administered to the patient.

This method is predicated on the discovery that when the concentration of deuterated arachidonic acid in the motor neurons is allowed to increase to a therapeutic level, the progression of the disease is significantly attenuated. The primer dose is continued for a period of at least up to about 30 days and, in some cases, at least up to about 45 days to ensure that the concentration of deuterated arachidonic acid reaches therapeutic levels.

Still further, when administered, 11,11-D2-linoleic acid requires enzymatic conversion of a portion of that PUFA to provide for 13,13-D2-arachidonic acid. The methods described herein are also predicated on the discovery that this conversion lags behind the time of administration of deuterated linoleic acid by several days. Indeed, a patient transitioning from the primer dose component to the maintenance dose continues to generate increased amounts of deuterated arachidonic acid well after initiation of the maintenance dose that utilized less deuterated linoleic acid or an ester thereof.

In one preferred embodiment, when dosing 11,11-D2-linoleic acid or an ester thereof, the primer dose in the methods described herein is sufficient to provide for a concentration of at least about 3 percent and preferably about 5 percent of 13,13-D2-arachidonic acid in the red blood cells within no more than about 45 days from initiation of treatment and preferably by about 30 days. The maintenance dose then maintains that percentage of 13,13-D2-arachidonic acid in motor neurons although with some tolerance such that the lower limit of no less than about 2.7 percent or so in the red blood cells.

In one embodiment, the primer dose of the 11,11-D2-linoleic acid or an ester thereof is preferably from about 7 to about 12 grams and is continued for at least about 24 days. The upper end for daily or periodic administration is determined by the attending clinician based on the degree of disease progression in the patient coupled with the patient's age, weight and other conditions and can run for up to about 60 days or longer (e.g, 180 days). In one preferred embodiment, the primer dose is continued from a period of from about 24 days up to about 45 days or any number of days or ranges therebetween.

This primer dose is used so as to provide rapid onset to the targeted therapeutic concentrations of 13,13-D2-arachidonic acid in the red blood cells and, by correlation, therapeutic concentration in the motor neurons. This primer dose is designed to compensate for variability in the patient's rate of metabolic conversion of 11,11-D2-linoleic acid to 13,13-D2-arachidonic acid. Preferred daily or periodic dosing of 11,11-D2-linoleic acid or ester thereof in the primer dose ranges from about 7 to about 12 grams per day and includes about 7 gm, about 7.5 gm, about 8 gm, about 8.5 gm, about 9 gm, about 9.5 gm, about 10 gm, about 10.5 gm, about 11 gm, about 11.5 gm, and about 12 gm.

As shown in Example 2, a primer dose of 9 grams of 11,11-D2-linoleic acid over a period of 30 days followed by a maintenance dose of 5 grams of 11,11-D2-linoleic acid provide for substantial reduction in the rate of disease progression.

The maintenance dose is initiated after completion of the primer dose and involves a reduced daily or periodic dose of 11,11-D2-linoleic acid or ester thereof. This reduced amount is described as a maintenance amount that provides for sufficient deuterated arachidonic acid in vivo to maintain the sufficient concentration of deuterated arachidonic acid in the spinal fluid and, hence, in the motor neurons. In general, the maintenance dose comprises about 30 to about 70 percent of the amount of 11,11-D2-linoleic acid or an ester thereof used in the primer dose. In one embodiment, the maintenance dose comprises about 35 to about 65 percent of the amount of deuterated linoleic acid or an ester thereof). In any case, the maintenance dose is less than the dosing dose.

In one embodiment, at the termination of the primer dose, the amount of 11,11-D2-linoleic acid or an ester thereof administered to the patient in the maintenance dose ranges from about 3 to about 6.5 gm per day. This reduced dosing of 11,11-D2-linoleic acid during the maintenance dose provides for maintenance of the targeted concentrations of 13,13-D2-arachidonic acid in the neurons of the patient. Preferred daily or periodic dosing of 11,11-D2-linoleic acid or ester thereof in the maintenance dose includes about 3 gm, about 3.5 gm, about 4 gm, about 4.5 gm, about 5 gm, about 5.5 gm, about 6 gm, and about 6.5 gm of 11,11-D2-linoleic acid or ester thereof.

In one embodiment, the patients are placed on a diet that restricts intake of excessive amounts of fats, including linoleic acid, arachidonic acid, and/or other PUFA compounds, to avoid insufficient uptake of the 11,11-D2-linoleic acid by the body. Generally, dietary components that contribute to excessive amounts of PUFA consumed are restricted. Such dietary components include, for example, fish oil pills, products that contain high levels of PUFAs such as salmon, and patients on conventional feeding tubes that result in excessive PUFA intake. In a preferred embodiment, the methods described herein include both the dosing regimen described above as well as placing the patients on a restrictive diet that avoids excessive ingestion of PUFA components.

In one embodiment, there is provided a method for reducing the rate of disease progression in a patient suffering from ALS which method comprises administering 11,11-D2-linoleic acid or an ester thereof to the patient with a dosing regimen that comprises a primer dosing and a maintenance dosing schedule which comprise:
a) said first dosing component comprises administering to said patient a primer dose of 11,11-D2-linoleic acid or an ester thereof in an amount to sufficient and for a period of time to allow for reduction in the rate of disease progression within no more than about 45 days from start of dosing;
b) subsequently following said primer dose, initiating a maintenance dosing to said patient said dosing comprises an amount of 11,11-D2-linoleic acid or an ester thereof in an amount sufficient to maintain the therapeutic concentration of 13,13-D2-arachidonic acid wherein the amount of 11,11-D2-linoleic acid or ester thereof administered in said maintenance dose is less than the amount administered in said primer dose; and optionally: and
c) monitoring the concentration of 13,13-D2-arachidonic acid in the patient to ensure that the patient is maintaining a therapeutic concentration of 13,13-D2-arachidonic acid.

In one embodiment, the clinician can increase the dosing of 11,11-D2-linoleic acid or an ester thereof when it is deemed that the concentration of 13,13-D2-arachidonic acid is deemed to be less than a therapeutic amount.

In one embodiment, the therapeutic amount of 13,13-D2-arachidonic acid in the neurons is determined by extrapolation from its concentration in red blood cells as provided herein. Such extrapolation requires that a patient being treated as per the methods herein is evaluated for the onset of a therapeutic result. When such a result is confirmed, the concentration of deuterated arachidonic acid in the red blood cells is assessed and that concentration is then used as a proxy for the therapeutic concentration.

Generally, based on this analysis, a concentration of 13,13-D2-arachidonic acid of about 3 percent in red blood cells based on the total amount of arachidonic acid, including deuterated arachidonic acid, therein is deemed to be therapeutic. However, when evaluating whether there should be an increase in the amount of 11,11-D2-linoleic acid or an ester thereof administered to the patient in the second component of the dosing schedule, the attending clinician can determine that a red blood concentration as low as about 2.7 percent is still therapeutic.

In one embodiment, there is provided a method for reducing the rate of disease progression in a patient suffering from ALS which method comprises administering 11,11-D2-linoleic acid or an ester thereof to the patient with a dosing regimen that comprises a primer dosing and a maintenance dosing schedule which comprise:
a) said primer dose comprises administering daily or periodically to said patient at least about 7 grams of 11,11-D2-linoleic acid or an ester thereof wherein said first daily or periodic dosing component is continued for a period of from about 24 to about 45 days to provide a therapeutic concentration of 13,13-D2-arachidonic acid in the motor neurons thereby reducing the rate of disease progression;
b) subsequently following said primer dose with a maintenance dose which comprises daily or periodic administration to said patient of about at least 3 grams of 11,11-D2-linoleic acid or an ester thereof to maintain a therapeutic concentration of 13,13-D2-arachidonic acid provided that said maintenance dose is less than said primer dose; and optionally:
c) monitoring the concentration of 13,13-D2-arachidonic acid in the patient to ensure that the patient maintains a therapeutic concentration of 13,13-D2-arachidonic acid; and
d) increasing the dosing of 11,11-D2-linoleic acid or an ester thereof when said concentrations of 13,13-D2-arachidonic acid are deemed to be less than a therapeutic concentration.

In one embodiment, there is provided a method for reducing the rate of disease progression in a patient suffering from ALS which method comprises administering 11,11-D2-linoleic acid or an ester thereof to the patient with a dosing regimen that comprises a primer dosing and a maintenance dosing schedule which comprise:
a) said first primer dose comprises about 9 grams of 11,11-D2-linoleic acid or an ester thereof wherein said daily or periodic primer dose is continued for about 30 days to 45 days, and preferably about 30 days, to provide a therapeutic concentration of 13,13-D2-arachidonic acid in the motor neurons thereby reducing the rate of disease progression;
b) subsequently following said daily or periodic primer dose with a daily or periodic maintenance dose of about 5 grams of 11,11-D2-linoleic acid or an ester thereof to maintain a therapeutic concentration of 13,13-D2-arachidonic acid in the motor neurons; and optionally
c) monitoring the concentration of 13,13-D2-arachidonic acid in the patient to ensure that the patient maintains a therapeutic concentration of 13,13-D2-arachidonic acid; and
d) increasing the dosing of 13,13-D2-arachidonic acid or an ester as well as a prodrug thereof when said concentrations of 13,13-D2-arachidonic acid are deemed to be less than a therapeutic concentration of 13,13-D2-arachidonic acid.

In another embodiment, the therapeutic concentration of 13,13-D2-arachidonic acid in the motor neurons correlates to a concentration of 13,13-D2-arachidonic acid of at least about 5% of the total amount of arachidonic acid, including deuterated arachidonic acid, in red blood cells.

In one embodiment, there is provided a kit of parts comprising a multiplicity of containers wherein each container contains a single daily or periodic dose of the first dosing component (e.g., 9 grams) or a single daily or periodic dose of the second dosing component (e.g., 5 grams). In one embodiment, each container comprises a plurality of dosing subunits. In one embodiment, each subunit comprises a pharmaceutical acceptable carrier and about 1 gm of 11,11-D2-linoleic acid such that the aggregate amount of 11,11-D2-linoleic acid in all of the subunits of a single container corresponds to either the first dosing component or the second dosing component.

In one embodiment, the aggregate of the subunits total 9 grams or 5 grams of 11,11-D2-linoleic acid.

In one embodiment, each container provides specific instructions to the patient as to his or her daily or periodic dosing including instructions to ingest all of the prescribed amount of drug.

In one embodiment, there is provided a unit dose of 11,11-D2-linoleic acid or an ester thereof comprising either about 9 grams or about 5 grams of 11,11-D2-linoleic acid or as ester thereof.

### BRIEF DESCRIPTION OF THE DRAWINGS

FIG. 1 is a graph showing the percent of 13,13-D2-Arachidonic Acid in red blood cells (RBC) and spinal fluid (SF) at the indicated time points after start of treatment with 11,11-D2-Linoleic Acid in an adult patient.

FIG. 2 is a graph showing the percent of 13,13-D2-Arachidonic Acid in red blood cells (RBC) and spinal fluid (SF) at the indicated time points after start of treatment with 11,11-D2-Linoleic Acid in juvenile patients.

### DETAILED DESCRIPTION

Disclosed are methods for dosing a patient with 11,11-D2-arachidonic acid in order to accelerate the time between initiation of therapy and achieving a therapeutic level of 13,13-D2-arachidonic acid in the neurons. In one embodiment, the methods described herein comprise first administering a primer dose to the patient which is continued for a sufficient period of time to achieve a therapeutic concentration of 13,13-D2-arachidonic acid in vivo. At that point, a maintenance dose is administered to maintain the therapeutic concentration of 13,13-D2-arachidonic acid.

In one embodiment, the methods also include limiting the dietary intake of polyunsaturated fatty acids in order to avoid limiting absorption of 11,11-D2-linoleic acid or ester thereof which could result in subtherapeutic concentrations of deuterated arachidonic acid in vivo.

Prior to discussing this disclosure in more detail, the following terms will first be defined. Terms that are not defined are given their definition in context or are given their medically acceptable definition.

The terminology used herein is for the purpose of describing particular embodiments only and is not intended to be limiting. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed methods.

As used herein, the term “consisting of” shall mean excluding more than trace elements of other ingredients and substantial method steps. Embodiments defined by each of these transition terms are within the scope of this disclosure.

As used herein, the term “ALS” refers to all forms of ALS including sALS and fALS.

As used herein, the term “linoleic acid” refers to the compound and a pharmaceutically acceptable salt thereof having the formula provided below and having the natural abundance of deuterium at each hydrogen atom:

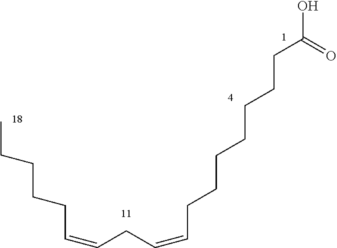 [SMILES: CCCCC/C=C\C/C=C\CCCCCCCC(=O)O]

Esters of linoleic acid are formed by replacing the —OH group with —OR. Such esters are as defined herein below.

As used herein and unless the context dictates otherwise, the term “deuterated linoleic acid or an ester thereof” refers to 11,11-D2-linoleic acid or esters thereof. Additional stabilization of the bis-allylic position could also include replacement of one or more of bis-allylic carbon atoms with a heavy isotope, alone or in conjunction with the deuteration (or tritiation), as the isotope effect (IE) resulting in stabilization of a bond with heavy isotopes is additive per long-established and fundamental chemical principles. (Westheimer, Chem. Rev. (1961), 61:265-273; Shchepinov, Rejuvenation Res., (2007), 10:47-59; Hill et al., Free Radic. Biol. Med., (2012), 53:893-906; Andreyev et al., Free Radic. Biol. Med., (2015), 82:63-72. Bigeleisen, J. The validity of the use of tracers to follow chemical reactions. Science, (1949), 110:14-16.

As used herein, arachidonic acid has the numbering system as described below:

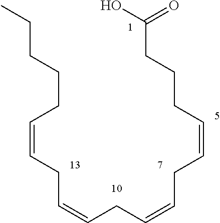 [SMILES: CCCCC/C=C\C/C=C\C/C=C\C/C=C\CCCC(=O)O]

where each of positions 7, 10 and 13 are bis-allylic positions within the structure.

As used herein and unless the context dictates otherwise, the term “deuterated arachidonic acid or an ester thereof” refers to 13,13-D2-arachidonic acid or ester compounds.

As used herein, the term “ester” means any pharmaceutically acceptable ester of a deuterated linoleic acid such as but not limited to C₁-C₆ alkyl esters, glycerol (including monoglycerides, diglycerides and triglycerides), sucrose esters, phosphate esters, and the like. The particular ester employed is not critical provided that the ester is pharmaceutically acceptable (non-toxic and biocompatible).

As used herein, the term “phospholipid” refers to any and all phospholipids that are components of the cell membrane. Included within this term are phosphatidylcholine, phosphatidylethanolamine, phosphatidylserine, and sphingomyelin. In the motor neurons, the cell membrane is enriched in phospholipids comprising arachidonic acid.

As used herein, the term “pathology of a disease” refers to the cause, development, structural/functional changes, and natural history associated with that disease. The term “natural history” means the progression of the disease in the absence of treatment.

As used herein, the term “reduced rate of disease progression” means that the rate of disease progression is attenuated after initiation of treatment as compared to the patient's natural history. In one embodiment, the reduced rate of disease progression is measured by using standardized tests such as the ALSFRS-R score to determine the rate of disease progression during the natural history and, again, measuring the score during the interval starting with therapy and ending at a set period of time thereafter (e.g., 6 months, 1 year, etc.). Both rates are then annualized and a reduced rate of disease progression results in a percentage change of at least 30% between the ALSFRS-R scores before and after.

A “therapeutic concentration” means a concentration of a deuterated arachidonic acid that reduces the rate of disease progression during therapy by at least 30% as compared to the rate of disease progression recorded for the natural history. Since obtaining the concentration of a deuterated arachidonic acid in the neurons or in the spinal fluid of a patient is either not feasible or optimal, the therapeutic concentration is based on the concentration of deuterated arachidonic acid found in red blood cells as provided in the Examples below. Accordingly, any reference made herein to a therapeutic concentration of deuterated arachidonic acid is made by evaluating its concentration in red blood cells.

Alternatively, the reduction in the rate of disease progression is confirmed by a reduction in the downward slope (flattening the curve) of a patient's relative functionality during therapy as compared to the downward slope found in the natural history. Typically, the differential between the downward slope measured prior to treatment and the slope measured after at least 90 days from initiation of treatment has a flattening level of at least about 30%. So, a change of 7.5 degrees (e.g., a downward slope of 25 degrees during the natural history that is reduced to a downward slope of 17.5 degrees provides for a 40% decrease in the slope). In any case, the reduction in downward slope evidence that the patient has a reduced rate of disease progression due to the therapy.

As used herein, the term “patient” refers to a human patient or a cohort of human patients suffering from ALS with an average of their disease progression being used.

As used herein, the term “loading or primer amount” refers to an amount of a deuterated linoleic acid or an ester thereof that is sufficient to provide for a reduced rate of disease progression within at least about 45 days after initiation of administration and preferably within 30 days. The amount so employed is loaded such that the patient has a stabilized rate of disease progression within this time period. When less than a loading amount is used, it is understood that such can provide therapeutic results but will not achieve the same level of reduction in disease progression. Given the progressive nature of neurodegenerative diseases, those dosing regimens that achieve the best reduction in the rate of disease progression are preferred as they are associated with the patient having less loss of functionality over a given period of time.

This disclosure includes the discovery that the primer doses of 11,11-D2-linoleic acid employed to date are well tolerated by patients and provide for rapid onset of a sufficient amount of deuterated arachidonic acid to provide for a reduced and stabilized rate of disease progression.

As used herein, the term “maintenance dose” refers to a dose of deuterated linoleic acid or an ester thereof or deuterated arachidonic acid that is less than the primer dose and is sufficient to maintain a therapeutic concentration of deuterated arachidonic acid in the cell membrane of red blood cells and, hence, in the cell membrane of motor neurons, that retains a stable rate of disease progression.

As used herein, the term “periodic dosing” refers to a dosing schedule that substantially comports to the dosing described herein. Stated differently, periodic dosing includes a patient who is compliant at least 75 percent of the time over a 30-day period and preferably at least 80% compliant contains a designed pause in dosing. For example, a dosing schedule that provides dosing 6 days a week is one form of periodic dosing. Another example is allowing the patient to pause administration for from about 3 or 7 or days due to personal reasons provided that the patient is otherwise at least 75 percent compliant.

The term “cohort” refers to a group of at least 2 patients whose results are to be averaged.

As used herein, the term “pharmaceutically acceptable salts” of compounds disclosed herein are within the scope of the present disclosure and include acid or base addition salts which retain the desired pharmacological activity and is not biologically undesirable (e.g., the salt is not unduly toxic, allergenic, or irritating, and is bioavailable). When the compound of the present disclosure has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present disclosure has an acidic group, such as for example, a carboxylic acid group, it can form salts with metals, such as alkali and earth alkali metals (e.g., Na⁺, Li⁺, K⁺, Ca²⁺, Mg²⁺, and Zn²⁺), ammonia or organic amines (e.g., dicyclohexylamine, trimethylamine, trimethylamine, pyridine, picoline, ethanolamine, diethanolamine, and triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

### Compound Preparation

11,11-D2-linoleic acid is known in the art. See, for example, U.S. Pat. No. 10,052,299 which is incorporated herein by reference in its entirety.

Esters of these deuterated fatty acids are prepared by conventional techniques well known in the art.

### Methodology—Deuterated Linoleic Acid

The methods of this disclosure utilize in vivo conversion of 11,11-D2-linoleic acid to 13,13-D2-arachidonic acid by administering 11,11-D2-linoleic acid or an ester thereof to a patient. When an ester is employed, hydrolysis of the ester to provide for the corresponding acid occurs in vivo as is bioconversion of the 11,11-D2-linoleic acid to 13,13-arachidonic acid.

In one embodiment, 11,11-D2-linoleic acid or ester thereof is administered to the patient in sufficient amounts to generate a concentration of 13,13-D2-arachidonic acid in red blood cells of at least about 3% based on the total amount of arachidonic acid, including deuterated arachidonic acid, present in the red blood cells.

In one embodiment, such administration comprises the use of a dosing regimen that includes two dosing components. The first dosing component comprises a primer dose of 11,11-D2-linoleic acid or an ester thereof. The second dosing component comprises a maintenance dose of 11,11-D2-linoleic acid or an ester thereof wherein the amount of 11,11-D2-linoleic acid or an ester thereof in said second dosing component is less than that of the first dosing component.

As to the primer dose, the amount of 11,11-D2-linoleic acid or an ester thereof employed is preferably designed to provide rapid onset of therapy. Such therapy is measured by a reduction in the disease progression as described below. The primer dose takes into account the various complicating factors such as the amount of PUFAs consumed by the patient in a given day, the in vivo rate of conversion of 11,11-D2-linoleic acid to 13,13-D2-arachidonic acid, as well as the general turnover rate of lipids in the motor neurons.

Regarding this last point, the lipid components of motor neurons are not static but, rather, are exchanged over time. In general, only a fraction of the lipids in the neurons are replaced each day. In the case of neurons, these cells are rich in lipids comprising arachidonic acid. The turnover of arachidonic acid in these membranes occurs from a stable pool of lipids comprising arachidonic acid in the spinal fluid. In turn, this stable pool is replaced and replenished over time by arachidonic acid included in the newly consumed lipids by the patient as part of the patient's diet as well as by biosynthesis of arachidonic acid from linoleic acid by the liver.

As to the latter, the rate of arachidonic acid synthesized in vivo is typically rate limited to the extent that there is a maximum amount of arachidonic acid that can be generated in a given day. In turn, only a fraction of the linoleic acid consumed is converted to arachidonic acid with the majority of the linoleic acid remaining unchanged. This limited rate of biosynthesis of arachidonic acid from linoleic acid results in a delay in such synthesis after administration of the deuterated linoleic acid as the amount of 13,13-D2-arachidonic acid concentration in red blood cells continues to increase after converting from the primer dose to the maintenance dose of the dosing regimen. This increase is contra-suggested as the maintenance dosing employs less 11,11-D2-linoleic acid as compared to the primer dose. However, without being limited to any theory, we believe that this increase is due to a lag in the in vivo conversion of 11,11-D2-linoleic acid to 13,13-D2-arachidonic acid after the administration of 11,11-D2-linoleic acid.

Hence, the choice of a dosing of 11,11-D2-linoleic acid must address each of the above components and set a dosing level that allows for the accumulation of a sufficient amount of 11,11-D2-linoleic acid in the body and, hence, the generation of therapeutic levels of 13,13-D2-arachidonic acid when measured in the proxy red blood cells. When so achieved, the data in the Examples establish that there is a significant reduction in the rate of disease progression.

Given the above, the dosing regimen described herein must include sufficient amounts of 11,11-D2-linoleic acid that are absorbed into the patient so as to maximize the in vivo conversion of 11,11-D2-linoleic acid 13,13-D2-arachidonic acid. Once maximized, the resulting deuterated arachidonic acid accumulates in the body until its concentration is stabilized in the lipid pool of the patient. Stabilization is reached, once the amount of deuterated 13,13-D2-arachidonic acid removed from the body is replaced by an equivalent amount of newly formed 13,13-D2-arachidonic acid. During this process, 13,13-D2-arachidonic acid is systemically absorbed into the cells of the body including neurons wherein the rate of which such absorption occurs is based on the exchange rate or turnover rate of lipids in the cell membrane of these neurons.

The methods described herein are based on the discovery that given the above variables, the amount of 11,11-D2-linoleic acid or ester thereof that is administered over time and converted in vivo to 13,13-D2-arachidonic acid is selected so that the red blood cells comprise at least about 3% and preferably at least about 5% of 13,13-D2-arachidonic acid. At that level, the deuterated arachidonic acid concentration stabilizes the cell membrane and limits or prevents the cascade of lipid auto-oxidation. When so administered, there is a significant reduction in the progression rate of the neurodegenerative disease.

The methods described herein are also based, in part, on the discovery that when the lipid membrane of neurons is stabilized against LPO, there is a substantial reduction in the progression of the neurodegenerative disease. This is due to the fact that replacement of hydrogen atoms with deuterium atoms at the bis-allylic positions of arachidonic acid renders the deuterated arachidonic acid significantly more stable to ROS than the hydrogen atoms. As above, this stability manifests itself in reducing the cascade of lipid auto-oxidation.

In the specific case of ALS, the reduction in the progression of this disease can be readily calculated by using the known and established rate functional decline measured by the R—ALS Functional Rating Scale-revised after commencement of drug therapy as compared to the rate of decline prior to drug therapy (natural history of decline). As the rate of decline is not perceptible on a day-to-day basis, the functional decline is typically measured monthly and is evaluated over a period of time such as every 3 months, every 6 months or annually.

As set forth in the examples below, the rate of functional decline is predicated on measuring an individual's but preferably a cohort's average for the natural history of disease progression. Next, the individual or cohort average for the functional decline is then determined at a period of time such as at 3, 6 or 12 months after initiation of therapy. The rate of decline based on the average of the natural history of the cohort is set as the denominator. The numerator is set as the delta between the rate of the natural history of disease progression and the rate of functional decline after a set period of treatment per this disclosure. The resulting fraction is then multiplied by 100 to give a percent change. The following exemplifies this analysis.

Cohort A has an average natural history rate of decline of 28 annualized for a one (1) year period. Six (6) months after initiation of treatment per this disclosure, Cohort A records an annualized average rate of decline of 14. This provides a delta of 14 degrees. So, using 14 as the numerator and 28 as the denominator and then multiplying result by 100, one obtains a reduction in the annualized rate of decline of 50 percent.

In general, the methods of this disclosure provide for an average percent reduction in functionality for a cohort of at least 30% and, more preferably, at least about 35%, or at least about 40%, or at least about 45%, or at least about 50%, or at least about 55%, or at least about 60% or more over either a 3 month, a 6 month, or annually. The rate of decline can be measured over any time period intermediate between 3 months and 1 year.

### Combinations

The therapy provided herein can be combined with conventional treatment of a given disease such as ALS provided that such therapy is operating on an orthogonal mechanism of action relative to inhibition of lipid auto-oxidation. Suitable drugs for use in combination include, but not limited to, anti-oxidants such as edaravone, idebenone, mitoquinone, mitoquinol, vitamin C, or vitamin E that are not directed to inhibiting lipid auto-oxidation, riluzole which preferentially blocks TTX-sensitive sodium channels, conventional pain relief mediations, and the like,

### Pharmaceutical Compositions

The specific dosing of a deuterated linoleic acid or an ester thereof (“drug”) administered to a patient can be accomplished by any number of the accepted modes of administration. As noted above, the actual amount of the drug used in a daily or periodic dose per the methods of this disclosure, i.e., the active ingredient, is described in detail above. The drug can be administered at least once a day, preferably once or twice or three times a day.

This disclosure is not limited to any particular composition or pharmaceutical carrier, as such may vary. In general, compounds of this disclosure will be administered as pharmaceutical compositions by any of a number of known routes of administration. However, oral delivery is preferred typically using tablets, pills, capsules, and the like. The particular form used for oral delivery is not critical but due to the large amount of drug to be administered, a daily or periodic unit dose is preferably divided into subunits having a number of tablets, pills, capsules, and the like. In one particularly preferred embodiment, each subunit of the daily or periodic unit dose contains about 1 gram of the drug. So, a daily or periodic unit dose of 9 grams of the drug is preferably provided as 9 sub-unit doses containing about 1 gram of the drug. Preferably, the unit dose is taken in one setting but, if patient compliance is enhanced by taking the daily or periodic unit dose over 2 or 3 settings per day, such is also acceptable.

Pharmaceutical dosage forms of a compound of this disclosure may be manufactured by any of the methods well-known in the art, such as, by conventional mixing, tableting, encapsulating, and the like. The compositions of this disclosure can include one or more physiologically acceptable inactive ingredients that facilitate processing of active molecules into preparations for pharmaceutical use.

The compositions can comprise the drug in combination with at least one pharmaceutically acceptable excipient. Acceptable excipients are non-toxic, aid administration, and do not adversely affect the therapeutic benefit of the claimed compounds. Such excipient may be any solid, liquid, or semi-solid that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Other suitable pharmaceutical excipients and their formulations are described in Remington's Pharmaceutical Sciences, edited by E. W. Martin (Mack Publishing Company, 18th ed., 1990).

The compositions of this disclosure may, if desired, be presented in a pack or dispenser device each containing a daily or periodic unit dosage containing the drug in the required number of subunits. Such a pack or device may, for example, comprise metal or plastic foil, such as a blister pack, a vial, or any other type of containment. The pack or dispenser device may be accompanied by instructions for administration including, for example, instructions to take all of the subunits constituting the daily or periodic dose contained therein.

The amount of the drug in a formulation can vary depending on the number of subunits required for the daily or periodic dose of the drug. Typically, the formulation will contain, on a weight percent (wt %) basis, from about 10 to 99 weight percent of the drug based on the total formulation, with the balance being one or more suitable pharmaceutical excipients. Preferably, the compound is present at a level of about 50 to 99 weight percent.

### EXAMPLES

This disclosure is further understood by reference to the following examples, which are intended to be purely exemplary of this disclosure. This disclosure is not limited in scope by the exemplified embodiments, which are intended as illustrations of single aspects of this disclosure only. Any methods that are functionally equivalent are within the scope of this disclosure. Various modifications of this disclosure in addition to those described herein will become apparent to those skilled in the art from the foregoing description and accompanying figures. Such modifications fall within the scope of the appended claims. In these examples, the following terms are used herein and have the following meanings. If not defined, the abbreviation has its conventional medical meaning.
D2-AA=13,13-D2-Arachidonic Acid
AA=Arachidonic Acid
ALSFRS-R=Revised ALS Functional Rating Scale
CNS=Central Nervous System
D2-LA=11,11-D2-Linoleic Acid
LA=Linoleic Acid
PK=Pharmacokinetics
RBC=Red Blood Cells
SAE=Serious Adverse Events
SF=Spinal Fluid
% deuterated PUFA=Percentage of a deuterated PUFA is based on the total amount of that PUFA, including the deuterated PUFA, present in the cell or medium being analyzed

### Example 1—AA Concentrations in RBCs and Spinal Fluid

This example was designed to evalutate how to calculate the concentration of D2-AA in the SF based on its concentration or the concentration of D2-linoleic acid in RBCs. Specifically, two separate correlations were done allowing for the calculation of either D2-LA or D2-AA in RBCs to provide a corresponding concentration of D2-AA in spinal fluid and, accordingly, in the motor neurons of the CNS. This proxy concentration allows the clinician a relatively facile means to measure the concentration of D2-AA in the spinal fluid without having to obtain spinal fluid from the patient.

In this example, a patient was continuously provided a daily dose of 9 grams of D2-LA ethyl ester over about a three-month period. Periodic samples of blood and SF were taken and the concentration of both D2-LA and D-2AA in both the RBCs and the SF were measured. In all cases, the D2-AA was obtained by deacylation of the ethyl ester followed by enzymatic conversion of D2-LA in vivo. Table 1 shows that the ratio of D2-LA: D2-AA in the SF at one month was about 2.5:1.

TABLE 1

| | Concentration | Concentration | Ratio of D2-LA |
|---|---|---|---|
| | of D2-LA | of D2-AA | to D2-AA |
| Time | in SF | in SF | in SF |
| 1 month | 19.8% | 8% | 2.5:1 |

Next, Table 2 shows that the concentration of D2-LA and D2-AA in the RBCs at 3 months and at 6 months. Here the ratio of D2-LA to D2-AA at 3 and 6 months is 2.5:1+/−0.4.

TABLE 2

| | Concentration | Concentration | Ratio of D2-LA |
|---|---|---|---|
| | of D2-LA | of D2-AA | to D2-AA |
| Time | in RBCs | in RBCs | in RBCs |
| 3 months | 34.7% | 11.8% | 2.9:1 |
| 6 months | 34.5% | 16.7% | 2.1:1 |

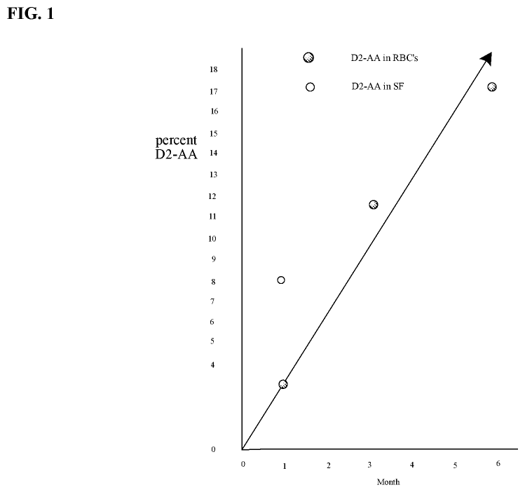

So, one can correlate that the concentration of D2-AA is about 2.5 times less than the concentration of D2-LA whether in RBCs or SF. Since the amount of D2-AA is increasing over time in an incremental fashion based on the conversion of D2-LA that limits the amount of D2-AA bio-generated per day, one can assume a fairly linear rate of increase. This is shown in FIG. 1 where the solid line is set by the concentrations of D2-AA at 3 months and 6 months and then extrapolated back to start of therapy (0 months). The value for the D2-AA in RBC's at 1 month is estimated from this linear relationship. The amount shown for 1 month in the SF is also provided (open circle).

Based on the above and FIG. 1, one can see that the data to date suggests that the amount D2-AA at 1 month in RBCs would be about 3 percent as compared to 8% for the amount of D2-AA in the SF. Accordingly, this data suggests that the concentration the body shunts more of the AA (including D2-AA) into the SF (and hence the neurons) as compared to RBCs and likely other reporter cells.

### Example 2—Stabilization of the Rate of Disease Progression

This example illustrates the reduction in the rate of disease progression in patients with ALS treated by the dosing methods of this disclosure. Specifically, a cohort of 3 patients was placed on a dosing regimen consisting of a first dosing component (primer dose) of about 9 grams of D2-LA ethyl ester daily for a period of at least 30 days and then all three patients were transitioned to a second dosing component (maintenance dose) of 5 grams of D2-LA ethyl ester.

The functionality of each of the patients was evaluated periodically using the ALSFRS-R protocol. The patients continued on the dosing regimen for a period of 6 months (patient A) or 1 year (patient B) or for 9 months (patient C). Patient C died at the end of 9 months and his death was attributed to factors other than ALS cardiomyopathy. Before initiation of therapy, the natural history of each patient in the cohort was determined and an average annual rate of functional decline was measured at 21.

The annualized progression of the disease as measured by an average annual rate of functional decline for all three patients starting at the time that dosing began and terminating at the end of the dosing regimen and then annualized as described above was measured as 2.1. Using the formula described above, one obtains the following:

(21−2.1)/21×100=90% annualized average reduction in the rate of disease progression.

The specific values for each of the three members of the cohort are as follows in Table 3:

TABLE 5

| | NH Rate | Functional Rate Decline |
|---|---|---|
| Patient | of Decline | During Therapy |
| A | −16 | −3 |
| B | −31 | −2 |
| C | −16 | −1.3 |

NH = Natural History

These results substantiate a very significant rate of reduction and stabilization in the disease progression using the dosing regimen as per this disclosure. These results also substantiate that transitioning patients from a primer dose to a maintenance dose maintains the beneficial stabilization in the rate of decline.

Even when the rate of decline for patient B is removed, the average rate of decline for the two remaining patients' natural history is −16 whereas the average rate of decline during the treatment period is 2.15. This provides for an 84% average rate of reduction in the disease progression. In addition, this example demonstrates that the tiered dosing protocol provides for exceptional reduction in the rate of disease progression.

### Example 3—Determination of Aa Concentrations in RBCS and Spinal Fluid/Neurons in a Cohort of 14 Patients

This example also determines the concentration of D2-AA in RBCs. Specifically, a cohort of 14 children was provided with a daily dose of 3 grams of D2-LA ethyl ester for months followed by 2 grams of D2-LA ethyl ester for the remaining six-month period. Blood samples were taken at 3 months for all but 1 child and at 6 months for all children. The concentration of D2-AA in RBCs was measured. In all cases, the D2-AA was obtained by deacylation of the ethyl ester of linoleic acid in the gastrointestinal tract followed by conversion of D2-LA in vivo to D2-AA.

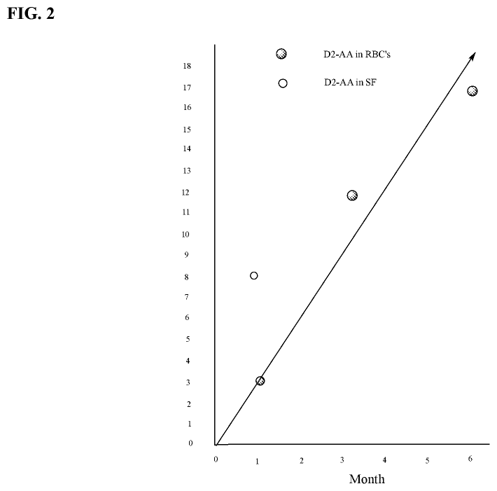

At 3 months, the average concentration of D2-AA in the RBCs was determined to be 12% (6.8% low and 16.8% high). At 6 months, the average concentration of D2-AA in the RBCs was determined to be 16.7% (12.0% low and 26.1% high). FIG. 2 depicts these results assuming a linear relationship of D2-AA accumulation in the body. Included in FIG. 2 is the 1-month data for D2-AA in the spinal fluid as found in Example 1.

As can be seen, FIGS. 1 and 2 are substantially the same strongly suggesting that the dosing of D2-LA to the adult patient in Example 1 and to the children in Example 3 maximized the conversion of D2-LA to D2-AA. This data further suggests that once maximized, the amounts of D2-AA generated over time are reproducible.

1. A method for accelerating uptake of 13,13-D2-arachidonic acid in vivo by dosing a patient with 11,11-D2-linoleic acid or an ester thereof in a patient wherein a portion of said 11,11-D2-linoleic acid is converted in vivo to 13,13-D2-arachidonic acid, the method comprising:
administering 11,11-D2-linoleic acid or an ester thereof to the patient with a dosing regimen that comprises a primer or loading dose followed by a maintenance dose, wherein:
a) said primer or loading dose comprises periodic administration of at least about 7 grams of 11,11-D2-linoleic acid or an ester thereof per day, wherein said primer or loading dose is continued for at least about 30 days to achieve a therapeutic concentration of 13,13-D2-arachidonic acid in vivo; and
b) subsequent to the completion of the primer or loading dose, administering said maintenance dose of from about 3 to about 6.5 gm per day of 11,11-D2-linoleic acid or an ester thereof per day thereof to maintain said therapeutic concentration of 13,13-D2-arachidonic acid in vivo.

2. A method for accelerating uptake of 13,13-D2-arachidonic acid in vivo by dosing a patient with 11,11-D2-linoleic acid or an ester thereof in a patient wherein a portion of said 11,11-D2-linoleic acid is converted in vivo to 13,13-D2-arachidonic acid, the method comprising:
administering 11,11-D2-linoleic acid or an ester thereof to the patient with a dosing regimen that comprises a primer or loading dose and a maintenance dose thereby reducing said disease progression in said patient, wherein:
a) said primer or loading dose comprises periodic administration at least about 7 grams of 11,11-D2-linoleic acid or an ester thereof per day, wherein said primer or loading dose is continued for at least about 30 days to rapidly achieve a therapeutic concentration of 13,13-D2-arachidonic acid in vivo; and
b) subsequent to the completion of the primer or loading dose, periodically administering said maintenance dose to said patient wherein said maintenance dose is at least about 3 grams per day of 11,11-D2-linoleic acid or an ester thereof per day thereof to maintain said therapeutic concentration of 13,13-D2-arachidonic acid in vivo provided that said maintenance dose is less than said primer dose.

3. A method for reducing disease progression of ALS in a patient, the method comprising:
administering an effective amount of 11,11-D2-linoleic acid to reduce said disease progression in said patient;
wherein a therapeutic concentration of 13,13-D2-arachidonic acid in the motor neurons is sufficient to reduce disease progression, wherein said reduction is measured to be at least about 30% reduction relative to the rate of disease progression during the natural history of the patient.

4. The method of claim 3, wherein the therapeutic concentration of deuterated arachidonic acid is determined by its concentration in red blood cells.

5. The method of claim 3, wherein a percent reduction in the rate of disease progression is determined by:
measuring a natural rate of disease progression in a patient or an average natural rate of disease progression in a cohort of patients;
measuring the rate of disease progression in said patient or cohort of patients during a period of compliance with the administering step; and
calculating the difference between the natural rate and the rate during the period of compliance, dividing the difference by the rate of disease progression during the natural history of the patient, and multiplying by 100.

6. The method of claim 3, wherein said deuterated linoleic acid or ester thereof is 11,11-D2-linoleic acid ethyl ester.

7. The method of claim 3, wherein the therapeutic concentration of 13,13-D2-arachidonic acid is determined by its concentration in red blood cells.

8. The method of claim 7, wherein the concentration of 13,13-D2-arachidonic acid in red blood cells is at least about 3% based on the total amount of arachidonic acid, including deuterated arachidonic acid, present in the red blood cells.

9. The method according to claim 3, wherein 11,11-D2-linoleic acid or an ester thereof is administered to the patient with a dosing regimen that comprises a primer dose and a maintenance dose, the method further comprising:
a) administering said primer dose to said patient at least about 7 grams of 11,11-D2-linoleic acid or an ester thereof wherein said first dosing component is continued for a period of from about 24 to about 45 days to provide a therapeutic concentration of 13,13-D2-arachidonic acid in red blood cells thereby reducing the rate of disease progression; and
b) subsequent to the period of from about 24 to about 45 days of administering said primer dose administering the maintenance dose to said patient, said maintenance dose comprising about at least 3 grams of 11,11-D2-linoleic acid or an ester thereof to maintain a therapeutic concentration of 13,13-D2-arachidonic acid provided that said maintenance dose is less than said primer dose.

10. The method of claim 9, which further comprises;
c) monitoring the concentration of 13,13-D2-arachidonic acid in the patient to ensure that the patient maintains a therapeutic concentration of 13,13-D2-arachidonic acid; and
d) increasing the dosing of 11,11-D2-linoleic acid or an ester thereof when said concentrations of 13,13-D2-arachidonic acid is deemed to be less than a therapeutic concentration.

11. The method of claim 10, which further comprises restricting the patient's consumption of excessive dietary polyunsaturated fatty acids during administration of said primer and said maintenance doses.

12. The method according to claim 3, wherein the method comprises administering 11,11-D2-linoleic acid or an ester thereof to the patient with a dosing regimen that comprises a primer dose and a maintenance dose:
a) said primer dose comprises about 9 grams of 11,11-D2-linoleic acid or an ester thereof wherein said primer dose is continued for about 30 days to 45 days to provide a therapeutic concentration of 13,13-D2-arachidonic acid thereby reducing the rate of disease progression; and
b) subsequent to the about 30 days to 45 days of administering said primer dose, administering said maintenance dose of about 5 grams of 11,11-D2-linoleic acid or an ester thereof to maintain a therapeutic concentration of 13,13-D2-arachidonic acid in the motor neurons.

13. The method of claim 12, which further comprises:
c) monitoring the concentration of 13,13-D2-arachidonic acid in the patient to ensure that the patient maintains a therapeutic concentration of 13,13-D2-arachidonic acid; and
d) increasing the dosing of 11,11-D2-linoleic acid or an ester when said concentrations of 13,13-D2-arachidonic acid is determined to be less than a therapeutic concentration.

14. The method of claim 13, which further comprises restricting the patient's consumption of excessive dietary polyunsaturated fatty acids during said first and second dosing components.

15. A kit of parts comprising a multiplicity of containers wherein each container contains a single daily dose of the primer dose totalling about 9 grams of 11,11-D2-linoleic acid or an ester thereof or a single daily dose of the maintenance dose totalling about 5 grams of 11,11-D2-linoleic acid.

16. The kit of parts of claim 15, wherein said primer dose comprises an aggregate of 9 subunits each subunit being about 1 gram.

17. The kit of parts of claim 15, wherein said maintenance dose comprises an aggregate of 5 subunits each subunit being about 1 gram.

18. A unit dose of 11,11-D2-linoleic acid or an ester thereof comprising either about 9 grams or about 5 grams of 11,11-D2-linoleic acid ethyl ester.
